(19) United States
(12) Patent Application Publication
(10) Pub. No.: US 2004/0087989 A1
(43) Pub. Date: May 6, 2004

(54) Scalpel sheath and methods for use

(75) Inventors: Rodney A. Brenneman, San Juan Capistrano, CA (US); Reynaldo B. Halili JR., Carlsbad, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: TheraCardia, Inc., San Clemente, CA (US)

(21) Appl. No.: 10/038,413

(22) Filed: Oct. 23, 2001

(51) Int. Cl. A61B 17/32

(52) U.S. Cl. 606/167

(57) ABSTRACT

The present invention provides improved devices and methods for making a small incision with a surgical scalpel through skin overlying an intercostal space to establish an access tract for the subsequent placement of minimally invasive direct cardiac massagers, chest tubes, defibrillation electrodes, and the like. A sheathed scalpel according to the present invention comprises a handle having a proximal end and a distal end, a cutting blade attached to the distal end of the handle and having a cutting edge and a tip, and a sheath attached to the handle and having a central passage configured to receive the blade. The sheath covers the blade when the blade is retracted within the central passage and exposes the cutting edge of the blade but not the tip when the blade is advanced within the central passage.

### BACKGROUND OF THE INVENTION

1. Field of the Invention

The present invention relates generally to medical devices and methods. More particularly, the present invention relates to improved devices and methods for making a small incision to establish a percutaneous intercostal access tract to a patient's heart for subsequent placement of minimally invasive direct cardiac massagers, chest tubes, defibrillation electrodes, and the like.

Sudden cardiac arrest is a leading cause of death in most industrial societies. In order to resuscitate a victim of cardiac arrest, it is necessary to provide an adequate artificial circulation of blood to oxygenate the heart and brain by re-establishing the pumping function of the heart during the period between cardiac arrest and restoration of normal cardiac activity. Such a cardiac pumping function must be instituted at the earliest possible state. While in many cases it is theoretically possible to re-establish cardiac function, irreversible damage to vital organs, particularly the brain and the heart itself, will usually occur if sufficient blood flow is not re-established within a short period of time from the moment of cardiac arrest.

A number of techniques have been developed to provide artificial circulation of blood to oxygenate the heart and brain during the period between cardiac arrest and restoration of normal cardiac activity. Prior to the 1960's, open chest cardiac massage (OCM) was a standard treatment for sudden cardiac arrest. Open chest cardiac massage, as its name implies, involved opening a patient's chest and manually squeezing the heart to pump blood to the body. In the 1960's, closed chest cardiac massage (CCM) where the heart is externally compressed through the chest wall became the standard of treatment. When CCM is combined with airway support, it is known as cardiopulmonary resuscitation (CPR). CPR has the advantage that it is much less invasive than OCM and can be performed by less skilled individuals. It has the disadvantage, however, that it is not generally effective. In particular, the medical literature shows that CCM provides significantly less cardiac output, neuroperfusion, and cardiac perfusion than achieved with OCM.

Of particular interest to the present invention is the recent introduction of devices for performing minimally invasive direct cardiac massage. Such devices and methods are described in co-pending application Nos. 09/087,665 filed May 29, 1998, now U.S. Pat. No. 6,200,280; 60/111,934 filed Dec. 11, 1998 (now abandoned); 09/344,440 filed Jun. 25, 1999; 09/356,064 filed Jul. 19, 1999; 09/801,421 filed Mar. 7, 2001; and 09/898,701 filed Jul. 2, 2001, assigned to the assignee of the present application. The fall disclosures of each of these prior patents and/or applications are incorporated herein by reference. Generally, such methods rely on introducing a plurality of struts, an expansible flared bell structure, a laterally oriented expansible structure, or other expandable member to engage the heart through a small incision through an intercostal space to a region over the pericardium or other heart surface. The heart may then be pumped by directly engaging the deployed expansible structure against the pericardium to repeatably compress the heart, typically by reciprocating a shaft attached to the member. Additional minimally invasive direct cardiac massage devices and methods are also described in 5,582,580; 5,571,074; and 5,484,391 issued to Buckman, Jr. et al. and U.S. Pat. Nos. 5,931,850; 5,683,364; and 5,466,221 issued to Zadini et al., licensed to the assignee of the present application.

While direct cardiac massage approaches offer great promise, one issue to be resolved for the success and practical utility of direct cardiac massage devices is establishing safe first entry into the chest cavity. Most methods of first entry employ a sharp surgical instrument, such as a scalpel, surgical knife, lancet, blade, and the like to make a small incision through skin overlying an intercostal space. However, the use of such sharp surgical scalpels is sometimes disadvantageous, particularly by less skilled treating individuals, as sharp dissection methods may result in serious risks, such as deep initial incisions from a single pass of the blade which may puncture and/or lacerate an organ, blood vessel, or surrounding structure. Moreover, due to concerns of safety against the transmission of diseases before and after the sharp instrument is used, many surgical scalpels have sheaths which permanently cover the blade component after a single use for disposal purposes. In certain circumstances, however, it may be desirable to have a blade that is readvancable as certain procedures may require a couple of blade passes to establish an intercostal access tract.

For these reasons, it would be desirable to provide improved devices and methods for making a small incision with a surgical scalpel to establish a percutaneous intercostal access tract to a patient's heart. In particular, it would be desirable to provide devices and methods which safely make a small incision through skin overlying an intercostal space to establish an access tract for the subsequent placement of minimally invasive direct cardiac massagers, chest tubes, defibrillation electrodes, and the like. The devices may be used by persons of minimal experience or training and may further be maintained safely and securely when not in use. At least some of these objectives will be met by the invention described hereinafter.

2. Description of the Background Art

Scalpel sheaths are described in U.S. Pat. Nos. 5,868,771; 5,665,099; 5,417,704; 5,330,492; 5,309,641; and 5,299,357. A protected disposable scalpel sold commercially by BD Bard-Parker™ is described at http://www.bd.com/surgical/surgical/scalpel.html. Devices and methods for minimally invasive direct cardiac massage through intercostal dissection are described co-pending U.S. patent application No. 09/087,665 filed May 29, 1998, now U.S. Pat. No. 6,200,280; U.S. Provisional Patent Application No. 60/111,934 filed Dec. 11, 1998 (now abandoned); U.S. patent application Nos. 09/344,440 filed Jun. 25, 1999; 09/356,064 filed Jul. 19, 1999; 09/801,421 filed Mar. 7, 2001; 09/895,844 filed Jun. 29, 2001; and 09/898,701 filed Jul. 2, 2001, assigned to the assignee of the present application. U.S. Pat. Nos. 5,484,391; 5,582,580; and 5,571,074 to Buckman, Jr. et al. and U.S. Pat. Nos. 5,466,221 and 5,683,364 to Zadini et al., licensed to the assignee of the present application, also describe devices and methods for minimally invasive direct cardiac massage through an intercostal space. Devices and methods for establishing intercostal access are described in co-pending U.S. patent application No. 09/768,041 filed Jan. 22, 2001, assigned to the assignee of the present application. U.S. Pat. No. 3,496,932 describes a sharpened stylet for introducing a cardiac massage device to a space between the sternum and the heart. Dissectors employing inflatable components are described in U.S. Pat. Nos. 5,730,756; 5,730,748; 5,716,325; 5,707,390; 5,702,417; 5,702,416; 5,694,951; 5,690,668; 5,685,826; 5,667,520; 5,667,479; 5,653,726; 5,624,381; 5,618,287; 5,607,443; 5,601,590; 5,601,589; 5,601,581; 5,593,418; 5,573,517; 5,540,711; 5,514,153; and 5,496,345.

The full disclosures of each of the above references are incorporated herein by reference.

### BRIEF SUMMARY OF THE INVENTION

The present invention provides improved devices and methods for making a small incision with a surgical scalpel to establish a percutaneous intercostal access tract to a patient's heart. In particular, the present invention provides devices and methods which safely make a small incision through skin overlying an intercostal space to establish an access tract for the subsequent placement of minimally invasive direct cardiac massagers, chest tubes, defibrillation electrodes, and the like. Moreover, the devices of present invention may be used by persons of minimal skill or training and may further be maintained safely and securely when not in use.

In a first aspect of the present invention, a sheathed scalpel comprises a handle having a proximal end and a distal end, a cutting blade attached to the distal end of the handle and having a cutting edge and a tip, and a sheath attached to the handle and having a central passage configured to receive the blade. The sheath covers the blade when the blade is retracted within the central passage and exposes the cutting edge of the blade but not the tip when the blade is advanced within the central passage. The sheath may have a structure disposed thereon to mate with a structure on the handle so as to prevent further advancement of the blade when the blade is advanced so that the blade tip remains covered. Additionally or alternatively, the sheath structure may mate with the handle structure so that the cutting edge of the blade is bowed and exposed though a sloping aperture of the central passage when the blade is fully advanced.

These structural features, alone or in combination, significantly reduce the risks of accidentally puncturing or cutting an organ, blood vessel, or surrounding structure. For example, a sheath that ensures that the blade tip remains covered in an advanced configuration protects against any deep stabbing or vertical cutting actions. A sheath that allows exposure of a bowed cutting edge of the blade through a sloping aperture in an advanced configuration limits an exposed blade area. Hence, such features act to reduce a cutting depth of the blade and to slow down the cutting action of the scalpel so that an initial small incision can be accurately and safely made though the skin in a couple of blade passes. The sheath further acts as a blade safety cover by fully covering the blade within the central passage when the scalpel is not in use so that risks and hazards associated with blade handling, such as disease transmission, are minimized.

The blade will have a cutting edge and a tip, wherein the blade tip has a leading end which may include but is not limited to a pointed or tapered tip. Typically, the bowed cutting edge of the blade will extend beyond the aperture by a depth in the range from 2 mm to 5 mm when the blade is fully advanced, preferably by a depth of about 3 mm. The blade and the handle may be two separate structures that are coupled together or preferably the blade is an integral extension of the handle.

The sheath will generally comprise an elongated housing structure that is preferably formed from a transparent or translucent material so that the sheath does not obstruct a treating person's view of the blade. The central passage has an aperture, hole, gap, slit, or opening that is diagonal relative to a longitudinal axis of the handle. The blade will generally be fixed relative to the handle and the sheath advancable and retractable relative to both the handle and the blade. Alternatively, the sheath may be fixed relative to the handle and the blade advancable and retractable relative to both the handle and the sheath.

It will be appreciated that advancement and retraction of the blade relative to the sheath may be limited by any number of conventional mechanisms. Typically, the central passage has an internal edge or slot near a distal end thereof and the handle has an edge which mates with the internal edge of the sheath to lock the blade and sheath into an advanced position. This prevents further blade advancement as well as ensures that the sheath does not slip or slide relative to the blade or handle during an incision procedure. The sheathed scalpel may further comprise a detent mechanism for limiting travel of the sheath relative to the blade. For example, the detent mechanism may comprise a series of axially spaced apart ribs or grooves on the handle which mate with a spring detent or transverse tab on the sheath when the blade is retracted to lock the blade securely within the sheath. The sheath may further comprise ribs on an inside surface that help to align the blade with the aperture and ribs or strips along an outside surface that facilitate gripping of the sheath relative to the blade or handle.

In a second aspect of the present invention, a sheath for use with a scalpel having a handle, a blade attached to the handle, and a structure on the handle, comprises an elongated housing having a central passage configured to receive the blade and an aperture. The housing aperture is disposed to expose a cutting edge of the blade when the blade is advanced within the central passage. When the blade is retracted within the housing, the housing fully covers the blade. The housing has a structure disposed thereon to mate with a structure on the handle so as to prevent further advancement of the blade when the blade is advanced so that a tip of the blade remains covered while a bowed cutting edge of the blade is exposed through the aperture.

In a third aspect of the present invention, methods for making a small incision through skin overlying an intercostal space are provided. One method comprises providing a scalpel having a sheath. The sheath is retracted relative to the scalpel so that a part of a scalpel blade is exposed beyond the sheath. The scalpel blade is then advanced through skin overlying the intercostal space to form the small incision, wherein a cutting depth of the scalpel blade through the overlying skin is limited by the sheath. A per pass cutting depth of the blade may be limited in several fashions. For instance, retracting may comprise exposing only a bowed cutting edge of the blade so that an exposed area of the scalpel blade is reduced. Preferably, the sheath will be retracted to provide a cutting depth in the range from 2 mm to 5 mm. Alternatively, retracting may comprise leaving a leading tip or end of the scalpel blade covered within the sheath. Retraction may be carried out by engaging an internal edge or slot of the sheath with an edge of a scalpel handle. The sheath may be easily retracted relative the scalpel handle with a single hand, wherein the force or pressure to retract the sheath is relatively small. Retraction may be facilitated by aligning the scalpel blade with an aperture of the sheath with ribs on an inside surface of the sheath.

Before and after blade use, the sheath may be extended over the scalpel blade so that the scalpel blade is entirely housed within the sheath for safety purposes. The extending typically comprises engaging an inner tab or spring detent on the sheath with at least one outer rib or groove on a scalpel handle. Moreover, in certain circumstances, it may be realized after an initial incision is made that it is not sufficient to establish an intercostal access tract. As such, the sheath of the present invention may be re-retracted relative to the scalpel so that the scalpel blade may be advanced at least a second time to make another cutting pass though the skin overlying the intercostal space. After an initial incision is made with the sheathed scalpel, a blunt member may then be advanced through the small incision and intercostal space above the heart to establish an intercostal access tract. The blunt member may comprise a gloved finger of a treating person, a blunt shaft or support, or like structure for clearing access to the heart and verifying the location of the heart. Following intercostal access establishment, a direct cardiac massage device may be advanced through the intercostal access tract. Exemplary cardiac massage devices are described in co-pending U.S. patent application No. 09/087,665 filed May 29, 1998, now U.S. Pat. No. 6,200,280; U.S. Provisional Patent Application No. 60/111,934 filed Dec. 11, 1998 (now abandoned); U.S. patent application Nos. 09/344,440 filed Jun. 25, 1999; 09/356,064 filed Jul. 19, 1999; 09/801,421 filed Mar. 7, 2001; and 09/898,701 filed Jul. 2, 2001, assigned to the assignee of the present application. Other suitable cardiac massage structures are described in U.S. Pat. Nos. 5,484,391; 5,582,580; and 5,571,074 issued to Buckman, Jr. et al. and 5,931,850; 5,683,364; and 5,466,221 issued to Zadini et al., licensed to the assignee of the present application.

A further understanding of the nature and advantages of the present invention will become apparent by reference to the remaining portions of the specification and drawings.

### BRIEF DESCRIPTION OF THE DRAWINGS

FIG. 1 is a perspective view of an exemplary sheathed scalpel constructed in accordance with the principles of the present invention.

FIGS. 2A and 2B are cross-sectional views of the sheath of FIG. 1.

FIGS. 3A and 3B are side views of the scalpel of FIG. 1.

FIGS. 4A-4B illustrate further top and cross-sectional views of the sheath of FIG. 1.

FIGS. 5A and 5B illustrate perspective and cross-sectional views of the scalpel blade in a retracted configuration.

FIGS. 6A and 6B illustrate perspective and cross-sectional views of the scalpel blade in an advanced configuration.

FIG. 7 is a cross-sectional view illustrating a heart underneath a patient's ribs.

FIGS. 8A-8E illustrate a method according to the present invention employing the device of FIG. 1.

FIG. 9 is a perspective view of a cardiac massage device used in conjunction with the present invention.

FIG. 10 illustrates a distal end of the cardiac massage device of FIG. 9, showing a deployed flared bell structure.

### DETAILED DESCRIPTION OF THE INVENTION

The present invention provides improved devices and methods for making a small incision with a surgical scalpel to establish a percutaneous intercostal access tract to a patient's heart. In particular, the present invention provides devices and methods which safely make a small incision through skin overlying an intercostal space to establish an access tract for the subsequent placement of minimally invasive direct cardiac massagers, chest tubes, defibrillation electrodes, and the like.

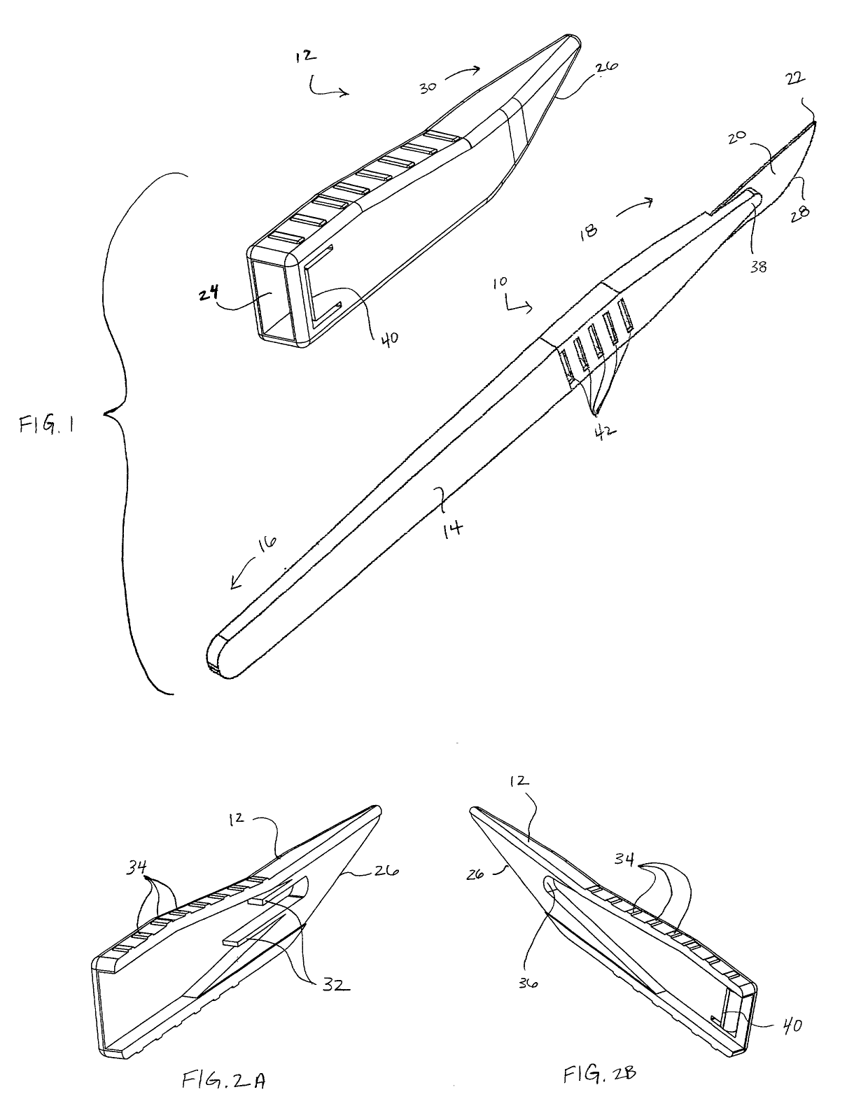

Referring now to FIG. 1, an exemplary sheathed scalpel constructed in accordance with the principles of the present invention for making a small incision through skin overlying an intercostal space is illustrated. A sheathed scalpel comprises a scalpel 10 and a sheath 12. The scalpel 10 has a handle 14 having a proximal end 16 and a distal end 18 and a cutting blade 20 having a cutting edge 28 and a tip 22 attached to the distal end 18 of the handle 12. The sheath 12 is attached to the handle 14 and has a central passage 24 configured to slidably receive the scalpel blade 20 and an aperture 26 disposed to expose the cutting edge 28 of the blade 20 but not the tip 22 when the blade 20 is advanced within the central passage 24. The sheath 12 also acts to fully cover the blade 20 when the blade 20 is retracted within the central passage 24. The sheath 12 has a structure disposed thereon to mate with a structure on the scalpel handle 14 so as to prevent further advancement of the blade 20 when the blade 20 is advanced so that the blade tip 22 remains covered. Additionally or alternatively, the sheath structure may mate with the handle structure so that a cutting edge 28 of the blade is bowed and exposed though a sloping aperture 26 when the blade 20 is advanced. It will be appreciated that the following depictions are for illustration purposes only and does not necessarily reflect the actual shape, size, or dimension of the sheathed scalpel. This applies to all depictions hereinafter.

The sheath 12 will generally comprise an elongated housing structure that has a relatively low profile and is contoured or tapered towards a distal end 30 to maintain an overall look and feel of a standard scalpel. The sheath 12 is preferably formed from a transparent or translucent material, such as a conventional polymer material including polyethylene, polyurethane, polystyrene, polycarbonate, polypropylene, and the like, so that the sheath does not obstruct a treating person's view of the blade 20. The sheath 12 has a length in the range from 2.5 inches to 3 inches, a width in the range from 0.3 inch to 0.6 inch, and a thickness in the range from 0.1 inch to 0.4 inch with a passage 24 or channel extending axially therethrough.

The scalpel handle 14 is of conventional shape and configuration, and is typically made of stainless steel, polyethylene, or other suitable material. Typically, a thickness of the handle increases in a distal direction to facilitate gripping of the scalpel. The scalpel blade 20 has a bowed cutting edge 28 and a tip 22, wherein the blade tip 22 has leading end which may include but is not limited to a pointed or tapered tip. Typically, the bowed cutting edge 28 will extend beyond the sheath aperture 26 by a depth in the range from 2 mm to 5 mm when the blade is fully advanced, preferably by a depth of about 3 mm. The scalpel blade 20 is preferably an integral extension of the handle 14. Preferably, the scalpel 10 is a Bard-Parker™ disposable #10 blade scalpel. It will be appreciated that the present invention described herein is intended to cooperate with a variety of scalpels and that sheath sizes, dimensions, and shapes may be varied accordingly.

Referring now to FIGS. 2A and 2B, the aperture 26, hole, gap, slit, or opening in the sheath 12 is preferably diagonal relative to a longitudinal axis of the handle. The sheath 12 has ribs 32 on an inside surface that help to align the blade 20 within the aperture 26, as shown in FIG. 2A. The sheath may have additional ribs or strips 34 along an outside surface that facilitate gripping of the sheath 12 relative to the scalpel 10. The sheath structure 36 may comprise an internal edge 36, as shown in FIG. 2B, tab, rib, button, aperture, or groove on or within the sheath 12.

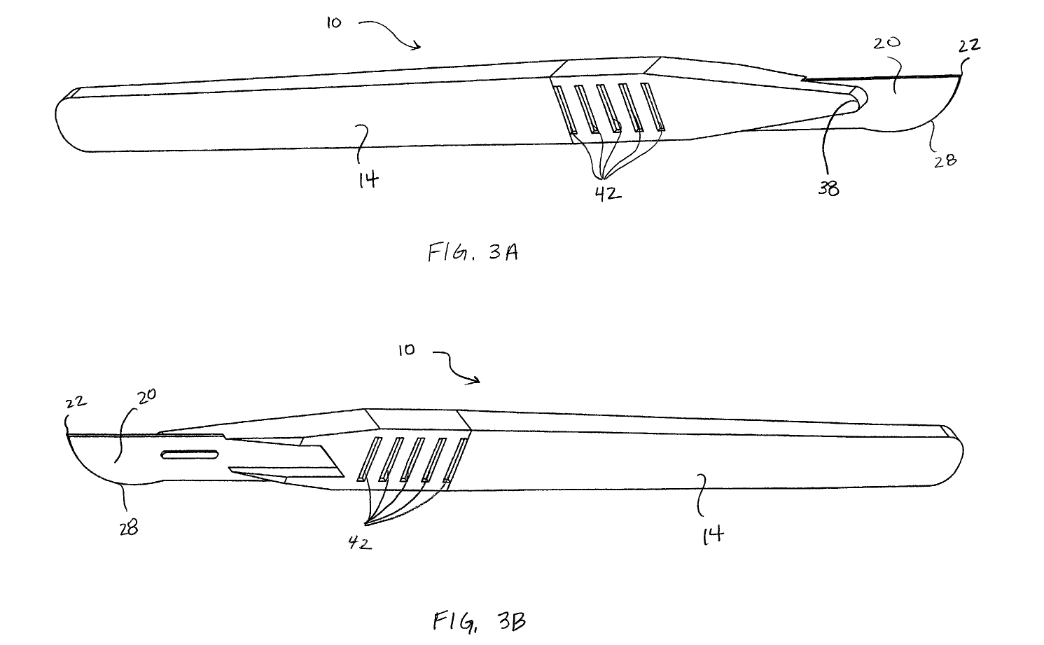

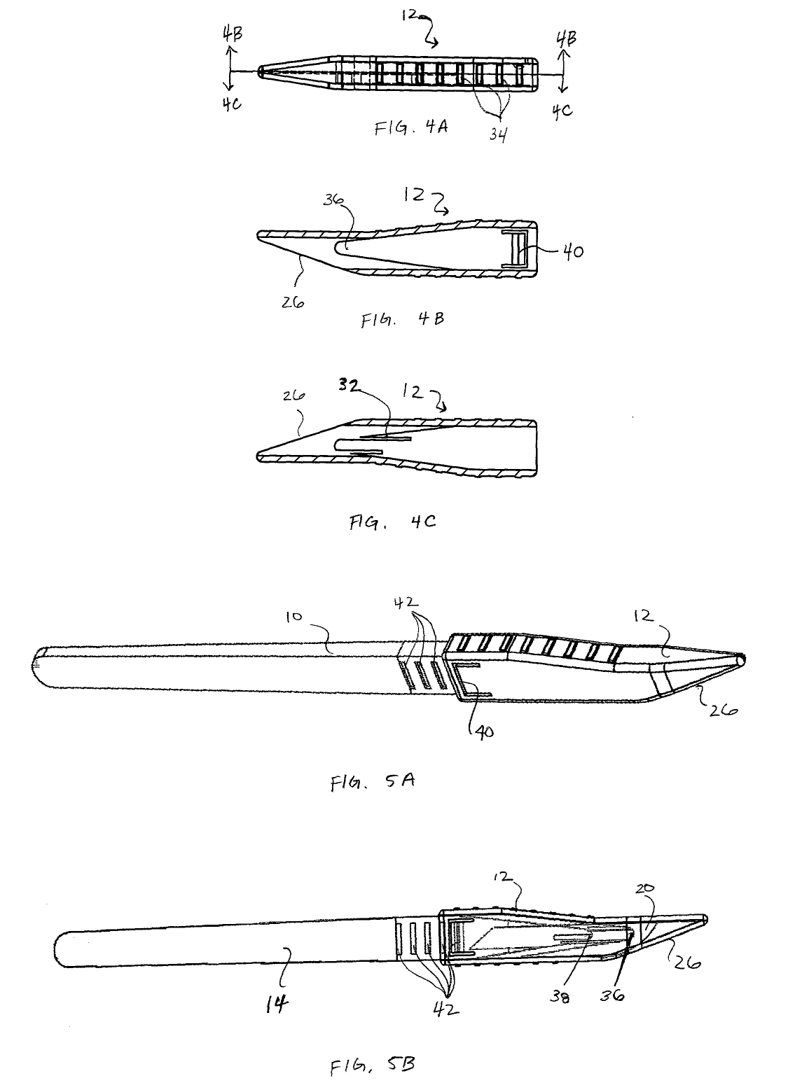

Referring now to FIGS. 3A and 3B, the handle structure 38 may comprise an edge 38, as depicted in FIG. 3A, tab, rib, button, aperture, or groove on the scalpel handle 14. Typically, the internal edge 36 of the sheath 12 mates with an edge 38 of the handle 14 to lock the blade 20 and sheath 12 into an advanced position. This interlocking mechanism prevents further blade advancement through the aperture 26 as well as ensures that the sheath 12 does not slip or slide relative to the blade 20 or handle 14 during an incision procedure.

Referring now to FIGS. 4A through 4C, further top and cross-sectional views of the sheath 12 are illustrated. A spring detent 40 or transverse tab on an inside surface of the sheath 12, as shown in FIG. 4B, mates with at least one rib 42 or groove on an outside surface of the scalpel handle 14 when the blade 20 is retracted. This interlocking mechanism securely locks the blade 20 within the sheath 12.

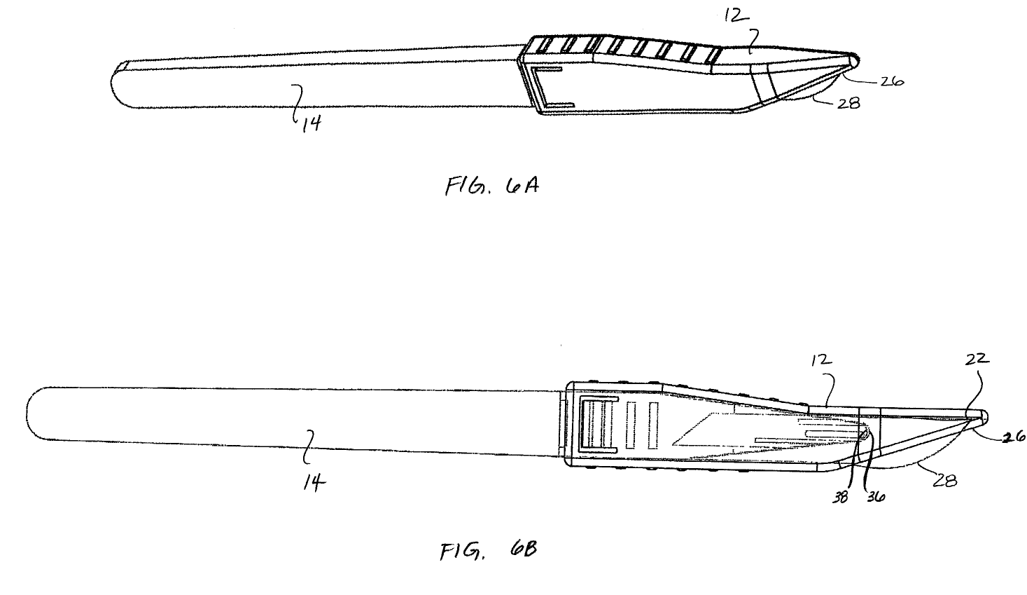

Referring now to FIGS. 5A and 5B, the scalpel blade in a retracted configuration is illustrated. As described above, advancement of the sheath over the blade is preferably limited by a detent mechanism, e.g. the sheath tab 40 interlocks with at least one handle rib 42 as shown in FIGS. 5A and 5B in safety position. As such, the sheath 12 acts as a blade safety cover when the scalpel 10 is not in use so that serous risks and hazards associated with blade handling, such as disease transmission, are minimized. FIGS. 6A and 6B illustrate the scalpel blade in an advanced configuration. In this position, the internal edge 36 (FIG. 2B) of the sheath 12 mates with an edge 38 (FIGS. 1 and 3A) of the handle 14. FIG. 6B shows that even when the blade cutting edge 28 is fully exposed the blade tip 22 remains covered within the sheath 12 to protect against any deep stabbing or vertical cutting actions. The sheath 12 allows exposure of only a bowed cutting edge 28 of the blade 20 through the sloping aperture 26 so as to limit an exposed blade area. Such structural features advantageously reduce a per pass cutting depth of the blade and slow down the cutting action of the scalpel so that an initial small incision can be accurately and safely made through the skin in a couple of cutting passes.

Referring now to FIG. 7, a patient's heart H is shown in a cross-section between ribs Rn where n indicates the rib number. The aorta A is also shown extending from the top of the heart.

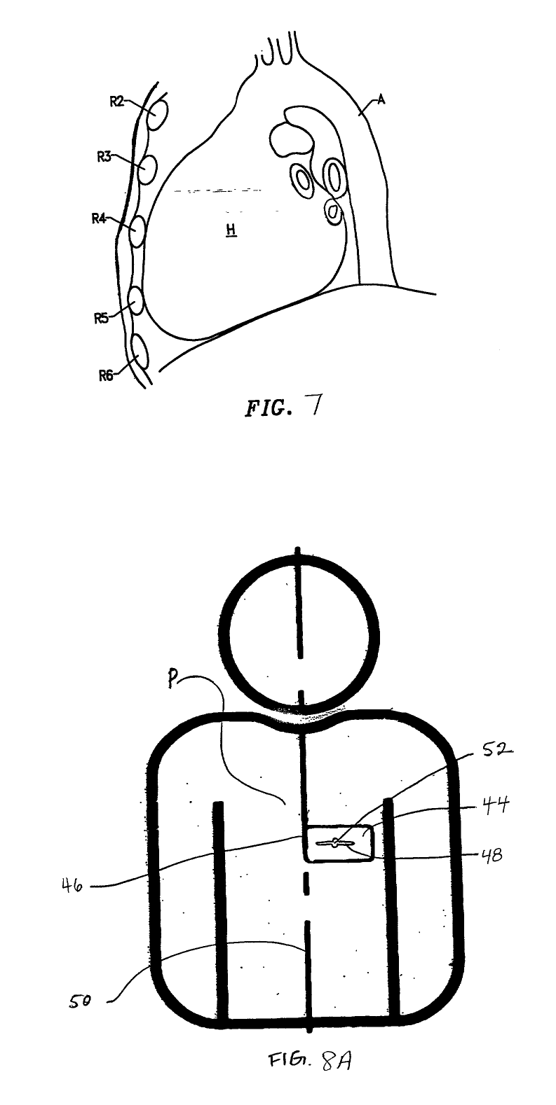

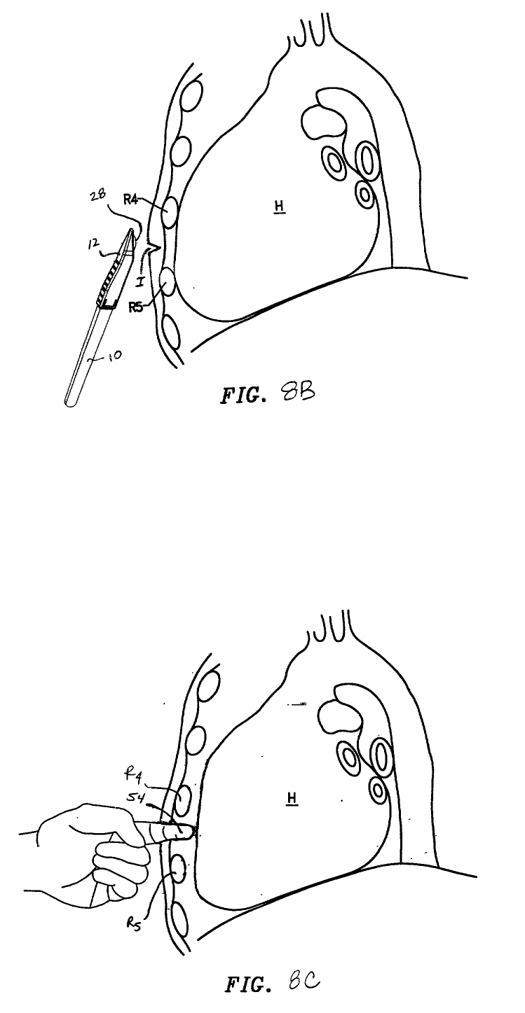

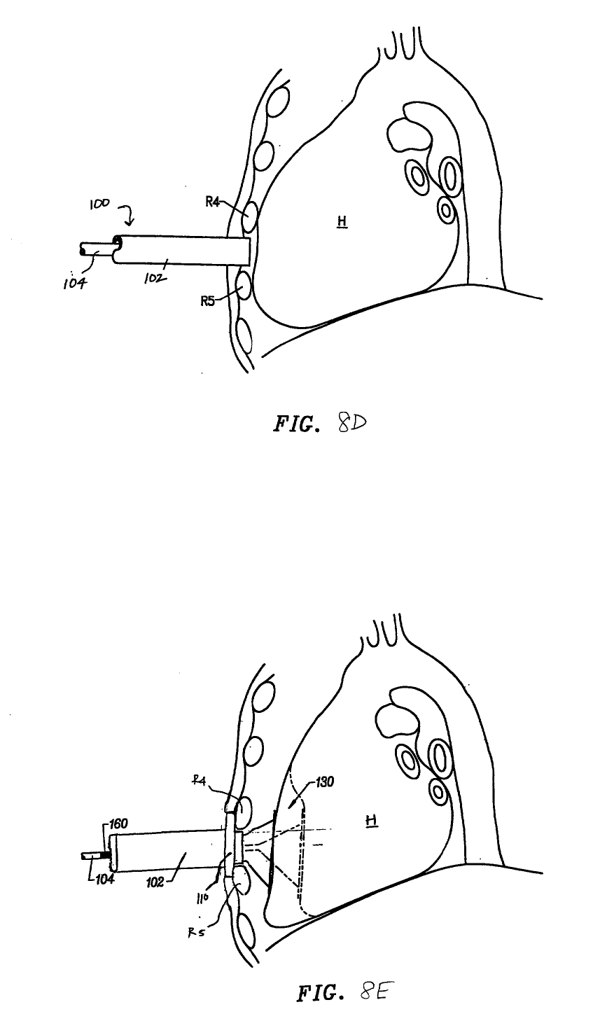

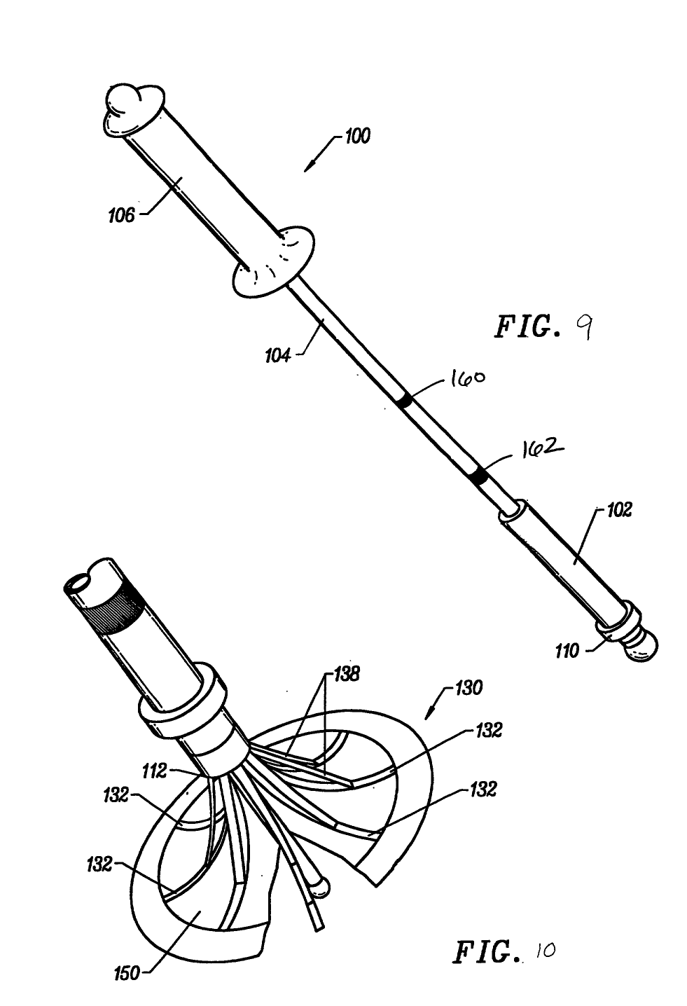

Referring now to FIGS. 8A through 8E, an exemplary method for making a small incision through skin overlying an intercostal space with the sheathed scalpel of FIG. 1 will be described. As illustrated in FIG. 8A, an incision template 44 (which is described in more detail in co-pending U.S. patent application No. 09/953,410 incorporated herein by reference) may be initially used to locate a site on a patient's chest suitable for establishing percutaneous intercostal access to the patient' heart H. At least one marker 46 on the template 44 is aligned with at least one anatomical feature of the patient P, the template 44 having a target zone opening 48 which lies over the site when the marker 46 is positioned with the anatomical feature. Preferably, a left edge or line 46 of the template 44 is aligned with a sternum mid-line 50 and a template opening 48 over a fourth intercostal space so that the target zone 48 (which also serves as a marker) lies over the site. In particular, the template opening 48 has a first axis which crosses with a second axis of the template opening to define an incision point 52 for subsequent entry. The incision point 52 will typically be located between ribs R₄ and R₅ of the patient, left of the mid-line sternum 50, and may be appropriately marked by a treating person with a surgical marker.

Referring now to FIG. 8B, a scalpel 10 having a sheath 12 is provided. The sheath 12 is retracted relative to the scalpel 10 so that a part of a scalpel blade 28 is exposed beyond the sheath 12. The scalpel blade 28 is then advanced through the incision point 52 defined by the incision template 44 to form the small incision I. A cutting depth of the scalpel blade through the overlying skin is limited by the sheath. A per pass cutting depth of the blade may be limited in several fashions. For example, retracting may comprise exposing only a bowed cutting edge 28 of the blade so that the exposed area of the scalpel blade is reduced. Preferably, the sheath 12 will be retracted to provide a cutting depth of the blade in the range from 2 mm to 5 mm. Alternatively, retracting may comprise leaving a leading tip 22 or end of the scalpel blade covered within the sheath 12. Retraction may be carried out by engaging an internal edge 36 of the sheath 12 with an edge 38 of a scalpel handle 14. The sheath 12 may be easily retracted relative the scalpel handle 14 with a single hand, wherein the force or pressure to retract the sheath is relatively small. Retraction may be facilitated by aligning the scalpel blade 20 with an aperture 26 of the sheath with ribs 32 on an inside surface of the sheath 12.

Before and after scalpel use, the sheath 12 may be extended over the scalpel blade 20 so that the scalpel blade 20 is entirely housed within the sheath 12 for safety purposes. The extending typically comprises engaging a spring detent 40 or tab on the sheath with at least one outer rib 42 on a scalpel handle. Moreover, in certain circumstances, it may be realized after an initial incision I is made that it is not sufficient to establish an intercostal access tract. As such, the sheath 12 of the present invention may be re-retracted relative to the scalpel 10 so that the bowed cutting edge 28 may be advanced at least a second time to make another cutting pass though the skin overlying the intercostal space. After an initial incision I is made with the sheathed scalpel, a blunt member 54 may then be advanced through the small incision and intercostal space above the heart to establish an intercostal access tract. The blunt member, in this case a gloved finger 54 of a treating person, clears access to the heart and verifies the location of the heart.

Following intercostal access establishment, a direct cardiac massage device 100 may be advanced as illustrated in FIGS. 8D and 8E. The cardiac massage device 100, as described in more detail in co-pending U.S. patent application Nos. 09/356,064 and 09/898,701, comprises a sleeve 102, a shaft 104 slidably mounted in a central lumen of the sleeve 102, and a handle 106 attached to a proximal end of the shaft (FIG. 9). The sleeve 102 includes a positioning flange 110 near its distal end, typically spaced proximally of a tip 112 of the device by an optimum distance. A flared bell structure 130, as best seen in FIG. 10, is attached to the distal end of shaft 104 and assumes a trumpeted configuration when fully deployed. The flared bell structure 130 comprises a plurality of outwardly curving struts 132 (the illustrated embodiment has a total of eight struts, but this number could vary). The struts are preferably formed from a resilient metal, usually formed from a superelastic alloy, such as nitinol. To enhance the rigidity and pushability of the structure, re-enforcing beams 138 may also be provided. It has been found that the combination of the curved struts with straight beam supports provides a useful combination of stiffness over the proximal portion of the structure and greater flexibility at the tip portions. The distal tips of the struts 130 are preferably connected by a fabric cover 150 having an edge which is folded over and stitched to hold the cover in place. The fabric cover may be a light mesh, composed of polyester or the like, and will help distribute forces quite evenly over the region of the pericardium which is contacted by the flared bell structure.

Turning back to FIGS. 8D and 8E, the device 100 is pushed through the incision until the flange 110 engages the ribs. Usually, the flared bell structure 130 will have a contracted profile configuration when introduced through the intercostal space. Once the structure is positioned to a region over a pericardium, the flared bell structure 130 is then deployed by advancing shaft 104 until a first marker 160 approaches the proximal end 162 of the sleeve 102. Once the structure 130 is fully deployed, the handle 106 may be manually grasped and the device shaft 104 pumped through the sleeve 102. This will cause the deployed flared bell structure 130 to compress the heart, generally shown in broken line in FIG. 8E. Once resuscitation has been completed, the device 100 may be withdrawn by retracting the shaft 104 relative to the sleeve 102 to draw the structure 130 back into the sleeve. The structure 130 will be sufficiently retraced as soon as the second marker 162 becomes visible out of the proximal end of the sleeve. Once the structure 130 is retracted, the device may be proximally withdrawn through the incision and the incision closed in a conventional manner.

Although certain preferred embodiments and methods have been disclosed herein, it will be apparent from the foregoing disclosure to those skilled in the art that variations and modification of such embodiments and methods may be made without departing from the true spirit and scope of the invention. Therefore, the above description should not be taken as limiting the scope of the invention which is defined by the appended claims.

What is claimed is:

1. A sheathed scalpel comprising:
a handle having a proximal end and a distal end;
a blade attached to the distal end of the handle and having a cutting edge and a tip; and
a sheath attached to the handle and having a central passage configured to receive the blade, wherein the sheath covers the blade when the blade is retracted within the central passage and exposes the cutting edge of the blade but not the tip when the blade is advanced within the central passage.

2. A sheathed scalpel as in claim 1, wherein the central passage has an aperture.

3. A sheathed scalpel as in claim 2, wherein the cutting edge of the blade is bowed and exposed though the aperture when the blade is advanced.

4. A sheathed scalpel as in claim 3, wherein the bowed cutting edge extends beyond the aperture by a depth in the range from 2 mm to 5 mm when the blade is fully advanced.

5. A sheathed scalpel as in claim 2, wherein the aperture is diagonal relative to a longitudinal axis of the handle.

6. A sheathed scalpel as in claim 1, wherein the sheath has a structure disposed thereon to mate with a structure on the handle so as to prevent further advancement of the blade when the blade is advanced so that the blade tip remains covered.

7. A sheathed scalpel as in claim 1, wherein the central passage has an internal edge near a distal end thereof and the handle has an edge which mates with the internal edge of the sheath so as to prevent further blade advancement.

8. A sheathed scalpel as in claim 1, further comprising a detent mechanism for limiting travel of the sheath relative to the blade.

9. A sheathed scalpel as in claim 8, wherein the detent mechanism comprises a series of axially spaced apart ribs or grooves on the handle and a spring detent or tab on the sheath.

10. A sheathed scalpel as in claim 1, further comprising ribs on an inside surface of the sheath.

11. A sheathed scalpel as in claim 1, wherein the sheath is transparent or translucent.

12. A sheathed scalpel as in claim 1, wherein the blade is fixed relative to the handle and the sheath advances and retracts relative to both the handle and the blade.

13. A sheathed scalpel as in claim 1, wherein the sheath is fixed relative to the handle and the blade advances relative to both the handle and the sheath.

14. A sheath for use with a scalpel having a handle, a blade attached to the handle, and a structure on the handle, the sheath comprising:
an elongated housing having a central passage configured to receive the blade and an aperture disposed to expose a cutting edge of the blade when the blade is advanced within the central passage, wherein the housing fully covers the blade when the blade is retracted within the housing;
wherein the housing has a structure disposed thereon to mate with a structure on the handle so as to prevent further advancement of the blade when the blade is advanced so that a tip of the blade remains covered while a bowed cutting edge of the blade is exposed through the aperture.

15. A method for making a small incision through skin overlying an intercostal space, the method comprising:
providing a scalpel having a sheath;
retracting the sheath relative to the scalpel so that a part of a scalpel blade is exposed beyond the sheath; and
advancing the scalpel blade through skin overlying the intercostal space to form the small incision, wherein a cutting depth of the scalpel blade through the overlying skin is limited by the sheath.

16. A method as in claim 15, wherein retracting comprises exposing only a bowed cutting edge of the blade so that an exposed area of the scalpel blade is reduced.

17. A method as in claim 15, wherein the sheath is retracted to provide a cutting depth of the blade in the range from 2 mm to 5 mm.

18. A method as in claim 15, wherein the retracting leaves a leading tip of the scalpel blade covered within the sheath.

19. A method as in claim 15, wherein retracting comprises engaging an internal edge of the sheath with an edge of a scalpel handle.

20. A method as in claim 15, further comprising aligning the scalpel blade with an aperture of the sheath with ribs on an inside surface of the sheath.

21. A method as in claim 15, further comprising extending the sheath over the scalpel blade so that the scalpel blade is housed within the sheath.

22. A method as in claim 21, wherein extending comprises engaging a spring detent or tab on the sheath with at least one outer rib or groove on a scalpel handle.

23. A method as in claim 21, further comprising re-retracting the sheath relative to the scalpel so that the scalpel blade may be advanced at least a second time.

24. A method as in claim 15, further comprising advancing a blunt member through the small incision and the intercostal space above the heart to establish an intercostal access tract.

25. A method as in claim 24, further comprising advancing a direct cardiac massage device through the intercostal access tract.
